(19) United States
(12) Patent Application Publication
(10) Pub. No.: US 2023/0240667 A1
(43) Pub. Date: Aug. 3, 2023

(54) MOBILE DEVICE FOR SALIVA COLLECTION

(71) Applicant: NATALIE SANDERS, Hampton, GA (US)

(72) Inventor: NATALIE SANDERS, Hampton, GA (US)

(21) Appl. No.: 18/161,390

(22) Filed: Jan. 30, 2023

Related U.S. Application Data
(60) Provisional application No. 63/304,734, filed on Jan. 31, 2022.

(51) Int. Cl. A61B 10/00; A61M 1/00

(52) U.S. Cl. CPC A61B 10/0051; A61M 1/71

(57) ABSTRACT

Disclosed are saliva collection containers comprising a removable cartridge containing a liquid absorbent material, the removable cartridge adapted to receive saliva from a user; a suction source removably disposed therein and fluidly coupled to the removable cartridge, wherein the saliva collection container is adapted to receive the removable cartridge; and a tubing having a first end and second end opposite of the first end, wherein the first end of the tubing is fluidly coupled to the suction source through an opening in the neck of the saliva collection container. Disclosed are saliva collection containers comprising a cartridge containing a liquid absorbent material, the cartridge adapted to fit inside a tubing; a tubing having a first end and second end opposite of the first end, wherein the first end of the tubing is fluidly coupled to the suction source; wherein the tubing is adapted to receive the cartridge; and a suction source removably disposed therein and fluidly coupled to the tubing.

### CROSS-REFERENCE TO RELATED APPLICATIONS

This application claims the benefit of U.S. Provisional Pat. Application No. 63/304,734, filed on Jan. 31, 2022, which is incorporated by reference herein in its entirety.

### BACKGROUND

Hypersalivation occurs when you have too much saliva or difficulty properly clearing the saliva you produce. Also known as sialorrhea or drooling, excess saliva can be temporary or a symptom of a chronic condition. For example, medications can cause hypersalivation, toxins can cause hypersalivation, neurological conditions can cause hypersalivation.

Medications can be taken to help treat hypersalivation but curing hypersalivation is not yet possible. Therefore, devices that help treat hypersalivation by removing excess saliva from a subject are needed.

### BRIEF SUMMARY

Disclosed are saliva collection containers comprising a removable cartridge containing a liquid absorbent material, the removable cartridge adapted to receive saliva from a user; a suction source removably disposed therein and fluidly coupled to the removable cartridge, wherein the saliva collection container is adapted to receive the removable cartridge; and a tubing having a first end and second end opposite of the first end, wherein the first end of the tubing is fluidly coupled to the suction source through an opening in the neck of the saliva collection container.

Disclosed are saliva collection containers comprising a cartridge containing a liquid absorbent material, the cartridge adapted to fit inside a tubing; a tubing having a first end and second end opposite of the first end, wherein the first end of the tubing is fluidly coupled to the suction source; wherein the tubing is adapted to receive the cartridge; and a suction source removably disposed therein and fluidly coupled to the tubing.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an example of the disclosed saliva collection containers in a housing unti.

FIG. 2 shows an example of the removable cartridge as a pouch comprising liquid absorbing material.

FIG. 3 shows an example of tubing entering the saliva collection container and connecting to the suction source.

FIG. 4 shows an example of a salive collection container comprising the cartridge in the tubing.

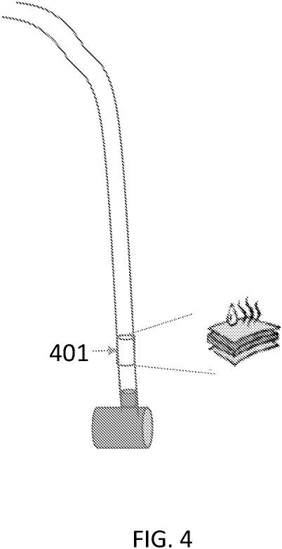

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a ”, “an”, and “the” include plural reference unless the context clearly dictates otherwise. Thus, for example, reference to “a tubing” includes a plurality of such tubings, reference to “the cartridge” is a reference to one or more cartridges and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Saliva Collection Container with Removable Cartridge Outside of Tubing

Disclosed are saliva collection containers comprising a removable cartridge containing a liquid absorbent material, the removable cartridge having a flow channel adapted to receive saliva from a user and transfer the saliva to the liquid absorbent material; a suction source removably disposed therein and fluidly coupled to the removable cartridge, wherein the saliva collection container is adapted to receive the removable cartridge and having a neck with an opening; a tubing having a first end and second end opposite of the first end, wherein the first end of the tubing is fluidly coupled to the suction source through the opening in the saliva collection container.

In some aspects, the saliva collection container comprises a body and tubing, wherein the body houses the removable cartridge, suction source, and a portion of the tubing.

In some aspects, the saliva collection container, specifically the body of the saliva collection container, comprises a neck with an opening.

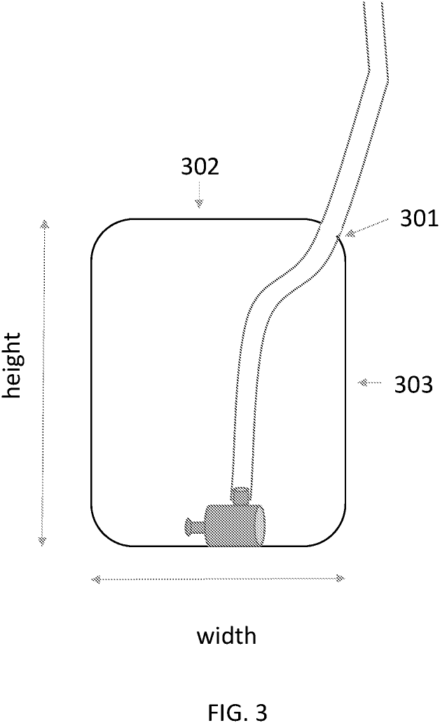

In some aspects, the size of the saliva collection container comprises a height of less 24 in, less than 12 in, less than 10 in, less than 8 in, less than 6 in, less than 4 in, wherein the height is measured as shown in FIG. 3. In some aspects, the size of the saliva collection container comprises a width of less than 12 in, less than 10 in, less than 8 in, less than 6 in, less than 4 in, less than 2 in, wherein the width is measured as shown in FIG. 3.

In some aspects, the shape of the saliva collection container can be, but is not limited to, square, rectangular, circular, or rounded. In some aspects, the saliva collection container

### 1. Removable Cartridge

The disclosed saliva collection containers comprise a removable cartridge containing a liquid absorbent material, the removable cartridge adapted to receive saliva from a user. In some aspects, the removable cartridge can transfer the saliva to the liquid absorbent material.

In some aspects, the removal cartridge has a flow channel adapted to receive saliva from a user.

In some aspects, the removable cartridge can be attached to the suction source. Thus, in some aspects, saliva is suctioned from the mouth, through the tubing, into the suction source and out of the suction source into the removable cartridge.

In some aspects, the removable cartridge is configured to attach to the suction source. In some aspects, a second piece of a tubing, referred to as a connector, can be used to connect the removable cartridge to the suction source. Specifically, the connector can attach to the outlet of the suction source and attach to the removable cartridge. In some aspects, the removable cartridge attaches directly to the suction source.

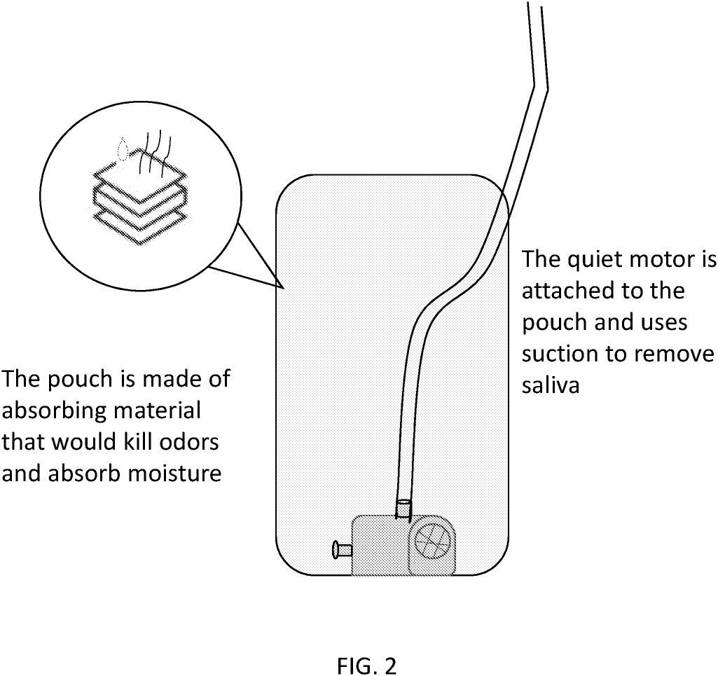

In some aspects, the removable cartridge is a pouch-like configuration containing a liquid absorbent material. In some aspects, the pouch-like configuration is connected to the suction source. In some aspects, the pouch-like configuration is not connected to the suction source. In some aspects, the suction source comprises an outlet, wherein the saliva flows through the suction source and comes out of the outlet directly into the pouch (FIG. 2).

In some aspects, the liquid absorbent material comprises highly absorbent clay called sodium bentonite or materials made from sodium polyacrylate, anhydrous calcium chloride, soda lime, allochroic silica gel, activated carbon, polystyrene, cotton, and/or baking soda. Any known liquid absorbent materials can be used. In some aspects, the liquid absorbent material can be in the form of sheets, powder, or pellets.

In some aspects, the liquid absorbent material further comprises odor reducing agents such as, but not limited to, baking soda, anti-bacterial agents, or essential oils. In some aspects, any known odor reducing agents can be used.

### 2. Suction Source

The disclosed saliva collection containers comprise a suction source removably disposed therein and fluidly coupled to the removable cartridge, wherein the saliva collection container is adapted to receive the removable cartridge.

In some aspects, the suction source can have an inlet and an outlet. In some aspects, the inlet receives the tubing and therefore is fluidly coupled to the tubing via the inlet. In some aspects, the outlet is fluidly coupled to the removable cartridge.

In some aspects, the suction source can be a vacuum pump. In some aspects, the suction source can be a high-volume suction source capable of servicing both LVE and HVE tools.

In some aspects, when the vacuum pump is activated, a suction of 5 to 125 Hg is applied. In some aspects, the suction is at least 5 Hg. In some aspects, the suction is at least 10 Hg. In some aspects, the suction is at least 15 Hg. In some aspects, the suction is at least 20 Hg. In some aspects, the suction is at least 50 mmHg, 100 mmHg, 150 mmHg, 200 mmHg, 250 mmHg, 300 mmHg, 350 mmHg, 400 mmHg, 450 mmHg, 500 mmHg, 550 mmHg, 600 mmHg, 650 mmHg, 700 mmHg, 750 mmHg, 800 mmHg, 850 mmHg, 900 mmHg, 950 mmHg, or 1000 mmHg.

In some aspects, the suction source has a suction of at least 100 L/min, 150 L/min, 200 L/min, 250 L/min, 300 L/min, or 350 L/min.

In some aspects, the suction source has a flow channel adapted to receive saliva from the tubing and transfer the saliva to the removable cartridge, particularly the liquid absorbent material in the removable cartridge. In some aspects, there is a filter that prevents liquid from entering the motor of the suction device device.

In some aspects, the noise level of the suction source is lower than 100, 90, 80, 70, 60 or 50 dB. In some aspects, vibration of the suction source is low.

In some aspects, the suction source can be controlled with an on/off switch 103. In some aspects, the on/off switch can be located on the suction source or on a housing unit that houses the suction source.

In some aspects, the suction source can be regulated to different strengths of suctioning. In some aspects, the suction source is controlled by a dial or switch that that can increase or decrease the suction. In some aspects, the suction source maintains a constant level of suction.

### 3. Tubing

The disclosed saliva collection containers comprise a tubing having a first end and second end opposite of the first end, wherein the first end of the tubing is fluidly coupled to the suction source through the opening in the neck of the saliva collection container. In some aspects, part of the tubing is contained on the inside of the saliva collection container. In some aspects, part of the tubing is present on the outside of the saliva collection container. In some aspects, the tubing present on the outside of the saliva collection container enters the saliva collection container via the opening in the neck of the saliva collection container allowing for part of the tubing to be contained on the inside of the saliva collection container.

In some aspects, the second end of the tubing comprises an opening. In some aspects, the second end of the tubing can be placed in the mouth and saliva is removed through the opening in the tubing. Thus, the opening at the second end of the tubing can be an opening through which saliva may be selectively drawn into the tubing.

In some aspects, the tubing can be bent at the second end, forming a hook or U-shape. This bend in the tubing can allow the tubing to bend over the lip.

In some aspects, the tubing can be standard high-volume evacuator (HVE), low-volume evacuator (LVE), saliva ejector (SE), or aspirator tubing.

In some aspects, the inner-bore diameter of the tubing can be any size. In some aspects, an inner-bore diameter of the tubing can range from ⅛″ up to 2″. In some aspects, the tubing can have any suitable wall thickness. In some aspects, the tubing can be smooth, corrugated, coiled, or any combination thereof, and can be substantially straight, curved, L-shaped, or in any other suitable linear configuration. In some aspects, the tubing can be made from extruded vinyl, silicone, polyvinyl chloride (PVC), nylon, or other suitable tubing material. In some aspects, the tubing can be made from biodegradable materials such as plant-based polymers, paper, and bamboo.

In some aspects, the tubing is clear. In some aspects, the tubing is not clear. Thus, in some aspects, the tubing is opaque or has a color. In some aspects, the tubing is not clear allowing for the saliva to not be seen traveling through the tubing.

In some aspects, the tubing has an inside diameter, wherein the inside diameter is constant throughout the length of the tubing. In some aspects, the inside diameter changes widths throughout the length of the tubing.

### 4. Opening

In some aspects, the saliva collection container comprises an opening 301. In some aspects, the opening can be anywhere within the saliva collection container. For example, in some aspects, the opening can be present at a top portion 302 or side portion 303 of the saliva collection container.

In some aspects, the opening is capable of receiving the tubing. In some aspects, the opening is slightly larger than the diameter of the tubing.

### 5. Mouthpiece

The disclosed saliva collection containers can optionally comprise a mouthpiece, wherein the mouthpiece is fluidly coupled to the second end of the tubing. Instead of using the opening at the second end of the tubing to place in the mouth, a mouthpiece fluidly coupled to the second end of the tubing can be what is placed in the mouth.

In some aspects, the mouthpiece can be a bite valve similar to what can be used with a CamelBak. In some aspects, a bite valve can be used so that suction is activated upon biting on the valve (e.g., mouthpiece). Thus, in some aspects, the mouthpiece is a self-sealing bite-actuated mouthpiece fluidly coupled to the second end of the tubing and including a sealable opening through which the saliva may be selectively drawn into the tubing, wherein the mouthpiece is selectively configured between a closed configuration, in which the mouthpiece is adapted to prevent saliva from being drawn therethrough, and a drawing configuration, in which the mouthpiece is adapted to permit saliva to be drawn therethrough via the suction source, wherein the mouthpiece is adapted to be configured from the closed configuration to the drawing configuration responsive to compressive forces applied thereto, and further wherein the mouthpiece is biased to automatically return from the drawing configuration to the closed configuration

In some aspects, the mouthpiece has several openings. In some aspects, the several openings allow saliva to enter into the tubing from all directions. In some aspects, the several openings are configured to prevent direct contact with the soft tissue in the mouth.

In some aspects, the mouthpiece can be that used in Comfort Plus® premium saliva ejectors.

In some aspects, the mouthpiece can be that of any known mouthpiece used in saliva ejectors in the dental industry.

### 6. Sensor

In some aspects, a sensor is optional. Thus, in some aspects, the disclosed saliva collection containers can comprise a sensor that indicates the saliva collection container is full. In some aspects, the saliva collection containers can be considered full once the liquid absorbent material is completely saturated.

In some aspects, a sensor is a change in color, a light that goes on or off, or a sound that goes off.

### 7. Housing Unit

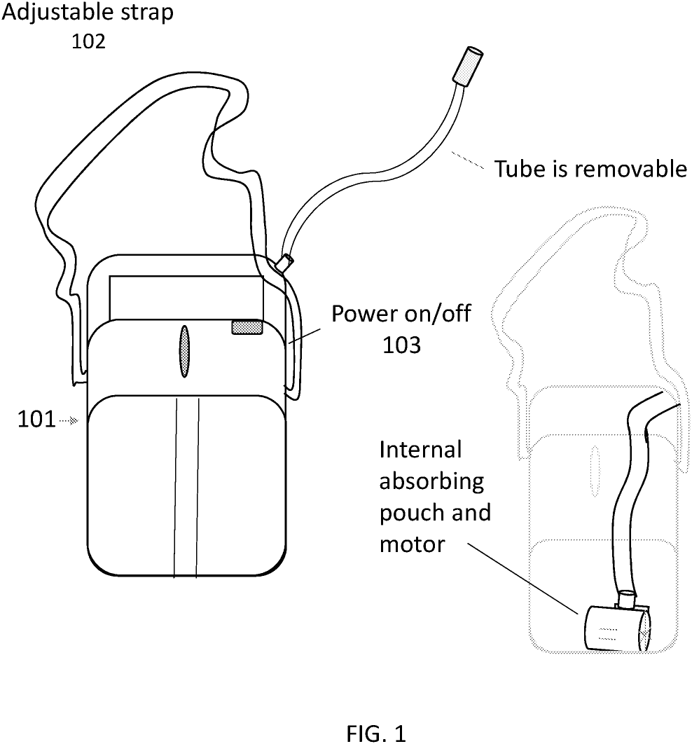

In some aspects, the saliva collection container is housed in a housing unit 101, for example, as shown in FIG. 1. In some aspects, the housing unit is big enough to hold the removable cartridge and suction source.

In some aspects, the housing unit can comprise pockets. In some aspects, the pockets can be used to store extra removable cartridges or tubing.

In some aspects, the housing unit comprises a carrying mechanism. In some aspects, the carrying mechanism is a fastening device, for example, but not limited to a clip. In some aspects, the clip can attach to another object, such as, the waistband of a garment, a pocket, a belt. In some aspects, the carrying mechanism is a handle, strap, or loop (all represented by 102). In some aspects, a loop can be used to attach the device to a belt.

### C. Saliva Collection Container with Cartridge Inside of Tubing

Disclosed are saliva collection containers comprising a cartridge containing a liquid absorbent material, the cartridge adapted to fit inside a tubing; a tubing having a first end and second end opposite of the first end, wherein the first end of the tubing is fluidly coupled to the suction source; wherein the tubing is adapted to receive the cartridge; and a suction source removably disposed therein and fluidly coupled to the tubing.

### 1. Cartridge

The disclosed saliva collection containers comprise a cartridge containing a liquid absorbent material, the cartridge adapted to fit inside a tubing. In some aspects, the cartridge can transfer the saliva to the liquid absorbent material.

In some aspects, the cartridge has a flow channel adapted to receive saliva from a user.

In some aspects, the cartridge is configured to fit within the tubing. Thus, in some aspects, the cartridge has a diameter smaller than the diameter of the tubing so that it can fit within the tubing. In some aspects, the cartridge can fill up one third, one half, or two thirds of the tubing. In some aspects, the cartridge can comprise a length of the tubing that is about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, or 12 inches.

In some aspects, the liquid absorbent material comprises highly absorbent clay called sodium bentonite or materials made from sodium polyacrylate, anhydrous calcium chloride, soda lime, allochroic silica gel, activated carbon, polystyrene, cotton, and/or baking soda. Any known liquid absorbent materials can be used. In some aspects, the liquid absorbent material can be in the form of sheets, powder, or pellets.

In some aspects, the liquid absorbent material further comprises odor reducing agents such as, but not limited to, baking soda, anti-bacterial agents, or essential oils. In some aspects, any known odor reducing agents can be used.

### 2. Tubing

The disclosed saliva collection containers comprise a tubing having a first end and second end opposite of the first end, wherein the first end of the tubing is fluidly coupled to the suction source; wherein the tubing is adapted to receive the cartridge.

In some aspects, the second end of the tubing comprises an opening. In some aspects, the second end of the tubing can be placed in the mouth and saliva is removed through the opening in the tubing. Thus, the opening at the second end of the tubing can be an opening through which saliva may be selectively drawn into the tubing.

In some aspects, the tubing is adapted to receive the cartridge. Thus, in some aspects, the cartridge is housed within the tubing. In some aspects, the cartridge can be anywhere within the tubing between the first end and the second end. In some aspects, the cartridge is closer to the first end than the second end.

In some aspects, the tubing can be bent at the second end, forming a hook or U-shape. This bend in the tubing can allow the tubing to bend over the lip.

In some aspects, the tubing can be standard high-volume evacuator (HVE), low-volume evacuator (LVE), saliva ejector (SE), or aspirator tubing.

In some aspects, the inner-bore diameter of the tubing can be any size. In some aspects, an inner-bore diameter of the tubing can range from ⅛″ up to 2″. In some aspects, the tubing can have any suitable wall thickness. In some aspects, the tubing can be smooth, corrugated, coiled, or any combination thereof, and can be substantially straight, curved, L-shaped, or in any other suitable linear configuration. In some aspects, the tubing can be made from extruded vinyl, silicone, polyvinylchloride (PVC), nylon, or other suitable tubing material. In some aspects, the tubing can be made from biodegradable materials such as plant-based polymers, paper, and bamboo.

In some aspects, the tubing is clear. In some aspects, the tubing is not clear. Thus, in some aspects, the tubing is opaque or has a color. In some aspects, the tubing is not clear allowing for the saliva to not be seen traveling through the tubing.

In some aspects, the tubing has an inside diameter, wherein the inside diameter is constant throughout the length of the tubing. In some aspects, the inside diameter changes widths throughout the length of the tubing.

### 3. Suction Source

The disclosed saliva collection containers comprise a suction source removably disposed therein and fluidly coupled to the tubing.

In some aspects, the suction source can have an inlet. In some aspects, the inlet receives the tubing and therefore is fluidly coupled to the tubing via the inlet. In some aspects, the suction source comprises a filter either within the inlet or in the tubing where the tubing connects to the suction source.

In some aspects, the suction source can be a vacuum pump. In some aspects, the suction source can be a high-volume suction source capable of servicing both LVE and HVE tools.

In some aspects, when the vacuum pump is activated, a suction of 5 to 125 Hg is applied. In some aspects, the suction is at least 5 Hg. In some aspects, the suction is at least 10 Hg. In some aspects, the suction is at least 15 Hg. In some aspects, the suction is at least 20 Hg. In some aspects, the suction is at least 50 mmHg, 100 mmHg, 150 mmHg, 200 mmHg, 250 mmHg, 300 mmHg, 350 mmHg, 400 mmHg, 450 mmHg, 500 mmHg, 550 mmHg, 600 mmHg, 650 mmHg, 700 mmHg, 750 mmHg, 800 mmHg, 850 mmHg, 900 mmHg, 950 mmHg, or 1000 mmHg.

In some aspects, the suction source has a suction of at least 100 L/min, 150 L/min, 200 L/min, 250 L/min, 300 L/min, or 350 L/min.

In some aspects, the noise level of the suction source is lower than 100, 90, 80, 70, 60 or 50 dB. In some aspects, vibration of the suction source is low.

In some aspects, the suction source can be controlled with an on/off switch 103. In some aspects, the on/off switch can be located on the suction source or on a housing unit that houses the suction source.

In some aspects, the suction source can be regulated to different strengths of suctioning. In some aspects, the suction source is controlled by a dial or switch that that can increase or decrease the suction. In some aspects, the suction source maintains a constant level of suction.

In some aspects, the saliva never enters the suction source as the saliva is absorbed within the cartridge prior to reaching the suction source.

### 4. Mouthpiece

The disclosed saliva collection containers can optionally comprise a mouthpiece, wherein the mouthpiece is fluidly coupled to the second end of the tubing. Instead of using the opening at the second end of the tubing to place in the mouth, a mouthpiece fluidly coupled to the second end of the tubing can be what is placed in the mouth.

In some aspects, the mouthpiece can be a bite valve similar to what can be used with a CamelBak. In some aspects, a bite valve can be used so that suction is activated upon biting on the valve (i.e. mouthpiece). Thus, in some aspects, the mouthpiece is a self-sealing bite-actuated mouthpiece fluidly coupled to the second end of the tubing and including a sealable opening through which the saliva may be selectively drawn into the tubing, wherein the mouthpiece is selectively configured between a closed configuration, in which the mouthpiece is adapted to prevent saliva from being drawn therethrough, and a drawing configuration, in which the mouthpiece is adapted to permit saliva to be drawn therethrough via the suction source, wherein the mouthpiece is adapted to be configured from the closed configuration to the drawing configuration responsive to compressive forces applied thereto, and further wherein the mouthpiece is biased to automatically return from the drawing configuration to the closed configuration

In some aspects, the mouthpiece has several openings. In some aspects, the several openings allow saliva to enter into the tubing from all directions. In some aspects, the several openings are configured to prevent direct contact with the soft tissue in the mouth.

In some aspects, the mouthpiece can be that used in Comfort Plus® premium saliva ejectors.

In some aspects, the mouthpiece can be that of any known mouthpiece used in saliva ejectors in the dental industry.

### 5. Sensor

In some aspects, a sensor is optional. Thus, in some aspects, the disclosed saliva collection containers can comprise a sensor that indicates the saliva collection container is full. In some aspects, the saliva collection containers can be considered full once the liquid absorbent material is completely saturated.

In some aspects, a sensor is a change in color, a light that goes on or off, or a sound that goes off.

### 6. Housing Unit

In some aspects, the saliva collection container is housed in a housing unit. In some aspects, the housing unit is big enough to hold the suction source.

In some aspects, part of the tubing is contained on the inside of the housing unit. In some aspects, part of the tubing is present on the outside of the housing unit.

In some aspects, the housing unit can comprise pockets. In some aspects, the pockets can be used to store extra removable cartridges or tubing.

In some aspects, the housing unit comprises a carrying mechanism. In some aspects, the carrying mechanism is a fastening device, for example, but not limited to a clip. In some aspects, the clip can attach to another object, such as, the waistband of a garment, a pocket, a belt. In some aspects, the carrying mechanism is a handle, strap, or loop (all represented by 102). In some aspects, a loop can be used to attach the device to a belt.

### D. Methods of Collecting Saliva

In some aspects, the disclosed devices can be used to help remove saliva from subjects suffering from hypersalivation. Hypersalivation can be caused by a variety of conditions. In some aspects, pregnancy can cause a condition called ptyalism gravidarum that causes hypersalivation. In some aspects, neurological conditions can cause excess saliva, these neurological conditions can include, but are not limited to, amyotrophic lateral sclerosis (ALS), Parkinson’s disease, cerebral palsy, stroke, and brain injury.

In some aspects, the disclosed devices can be used in combination with a known medical treatment for hypersalivation, such as but not limited to, anticholinergic medications, botox injections, radiation, Salivary gland ablation or surgery. For example, anticholinergic medications can block activity in the salivary glands; botox injections in the salivary glands can decrease saliva production; radiation can cause dry mouth thus reducing saliva; salivary gland ablation involves injections of chemicals to cause salivary glands to shrink and become scar tissue; and surgery can be used to reroute salivary ducts, close them off, or remove salivary glands.

In some aspects, the second end of the tubing, or mouthpiece, can be placed in a subject’s mouth and saliva can be removed from the subject’s mouth. The suction source can be turned on before or after placing the tubing in the subject’s mouth.

Those skilled in the art will recognize, or be able to ascertain using no more than routine experimentation, many equivalents to the specific embodiments of the method and compositions described herein. Such equivalents are intended to be encompassed by the following claims.

1. A saliva collection container comprising:
a removable cartridge containing a liquid absorbent material, the removable cartridge adapted to receive saliva from a user:
a suction source removably disposed therein and fluidly coupled to the removable cartridge, wherein the saliva collection container is adapted to receive the removable cartridge: and
a tubing having a first end and second end opposite of the first end, wherein the first end of the tubing is fluidly coupled to the suction source through an opening in the saliva collection container.

2. The saliva collection container of claim 1, wherein the saliva collection container further comprises a mouthpiece fluidly coupled to the second end of the tubing.

3. The saliva collection container of claim 2, wherein the mouthpiece is a self-sealing bite-actuated mouthpiece comprising a sealable opening through which the saliva may be selectively drawn into the tubing, wherein the mouthpiece is selectively configured between a closed configuration, in which the mouthpiece is adapted to prevent saliva from being drawn therethrough, and a drawing configuration, in which the mouthpiece is adapted to permit saliva to be drawn therethrough via the suction source, wherein the mouthpiece is adapted to be configured from the closed configuration to the drawing configuration responsive to compressive forces applied thereto, and further wherein the mouthpiece is biased to automatically return from the drawing configuration to the closed configuration.

4. The saliva collection container of claim 2, wherein the mouthpiece comprises a soft tip covering an opening at the second end of the tubing.

5. The saliva collection container of claim 2, wherein the mouthpiece comprises two or more openings.

6. The saliva collection container of claim 2, wherein the mouthpiece is rounded.

7. The saliva collection container of claim 2, wherein the mouthpiece comprises a filter.

8. The saliva collection container of claim 1, wherein the tubing has an inside diameter wherein the inside diameter is constant throughout the tubing.

9. The saliva collection container of claim 1, wherein the suction source comprises a motor.

10. The saliva collection container of claim 1, wherein the saliva collection container is disposable.

11. The saliva collection container of claim 1, wherein the liquid absorbent material comprises sodium bentonite.

12. The saliva collection container of claim 1, wherein the liquid absorbent material comprises an odor reducing agent.

13. The saliva collection container of claim 12, wherein the odor reducing agent is baking soda, an anti-bacterial agent, or an essential oil.

14. The saliva collection container of claim 1, wherein the suction source can be regulated to different strengths of suctioning.

15. The saliva collection container of claim 1, wherein the tubing is not clear.

16. The saliva collection container of claim 1, wherein the saliva collection container is housed in a housing unit.

17. The saliva collection container of claim 16, wherein the housing unit comprises a carrying mechanism.

18. The saliva collection container of claim 17, wherein the carrying mechanism is a fastening device.

19. (canceled)

20. The saliva collection container of claim 17, wherein the carrying mechanism is a handle, strap, or loop.

21. (canceled)

22. A saliva collection container comprising:
a cartridge containing a liquid absorbent material, the cartridge adapted to fit inside a tubing;
a tubing having a first end and second end opposite of the first end, wherein the first end of the tubing is fluidly coupled to the suction source; wherein the tubing is adapted to receive the cartridge: and
a suction source removably disposed therein and fluidly coupled to the tubing.
